(19) United States
(12) Patent Application Publication
(10) Pub. No.: US 2005/0119672 A1
(43) Pub. Date: Jun. 2, 2005

(54) Gastric ring made of variable hardness elastomeric material

(76) Inventor: Salomon Benchetrit, Caluire et Cuire (FR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/501,645

(22) PCT Filed: Nov. 15, 2002
(86) PCT No.: PCT/FR02/03933

(30) Foreign Application Priority Data
Jan. 15, 2002 (FR) 02/00517

(51) Int. Cl. A61B 17/08

(52) U.S. Cl. 606/151; 606/157

(57) ABSTRACT

A gastroplasty band, as disclosed herein, comprises a flexible strip designed to be closed around the stomach of a patient by a closure mechanism. The flexible strip includes an annular compression chamber connected by a catheter to a device for adjusting the diameter of the compression chamber by injecting or withdrawing fluid. The compression chamber comprises a dorsal reinforcement extended by lateral walls. The dorsal reinforcement is made out of a first elastomer material having predetermined hardness d₁ on the Shore A scale. The lateral walls are made out of a second elastomer material of the same kind as the first elastomer material but of predetermined hardness d₂ on the Shore A scale such that d₂<d₁. The annular compression chamber thus has a hardness on the Shore A scale that varies across its thickness.

### TECHNICAL FIELD

The present invention relates to the general technical field of surgical implants for treating obesity by implanting a flexible gastric strip for closing around the stomach of a patient in order to reduce the diameter of the opening of the stoma so as to constrain the patient to reduce food intake.

The present invention relates to a gastroplasty band formed by a flexible strip designed to be closed around the stomach of a patient by closure means close to the two ends of the strip, thereby reducing the diameter of the opening of the stoma, said strip including an annular compression chamber of adjustable volume connected by a catheter to a device for adjusting the diameter of said chamber by injecting or withdrawing fluid, said chamber being defined by walls that include dorsal reinforcement extended by lateral walls.

### PRIOR ART

For patients suffering from extremely severe obesity (morbid obesity), i.e. patients whose weight generally exceeds their ideal weight by at least 50 kilograms (kg), for example, it is absolutely necessary to intervene surgically in order to avoid not only severe health problems but also to avoid almost certain death of such patients in the near term.

It is accepted that patients suffering from morbid obesity have their life expectancy reduced considerably, by at least ten to fifteen years, while also leading to severe psychological problems. Furthermore, collateral health problems are generally also seen to appear, such as the appearance of cardiovascular diseases or the appearance of phenomena associated with hypertension, diabetes, and severe arthritis, in particular.

It is also known that with extremely severe obesity, conventional curative treatments based on a severe diet, e.g. combined with a series of physical exercises, have little effect on such cases of extreme obesity.

That is why effective and long-term treatment of morbid obesity involves surgical treatment.

In general, a distinction is drawn between surgical treatments that involve reducing the extent to which food is absorbed, i.e. shortening the conventional path followed by food and digestive juices, and techniques that involve gastric restriction, i.e. reducing the size of the stomach.

Surgical techniques involving reduced absorption are, for example, techniques in which a bypass of the small intestine is made or techniques that involve separating the food passage from that of digestive juices. Those techniques are nowadays rarely used since they can lead to severe complications for the patient and in all cases they involve a large amount of surgery.

That is why the present trend is to favor surgical techniques that involve gastric restriction for reducing food intake.

These well-known techniques make use of gastroplasty bands implanted around the stomach of a patient in order to reduce the size of the stomach and the diameter of the passage through it (the stoma).

The general structure of the gastroplasty bands used is well known and involves a flexible strip made of elastomer material for closing around the stomach of a patient by closure means located towards the two ends of the strip, thereby reducing the diameter of the opening in the stoma. The closure means are generally situated on the outer or dorsal portion of the flexible strip and involve various types of locking, e.g. mechanical locking with or without suturing. Known bands also include a strip having an annular compression chamber of volume or diametral expansion that is adjustable, said chamber being suitable for being connected by a catheter to a device for adjusting the diameter of the chamber by injecting or withdrawing fluid. By means of this feature, it is possible, starting from a band of fixed size or diameter, to adjust the diameter of the band finely by injecting or withdrawing fluid, thereby leading to a corresponding increase or reduction in the diameter of the band.

The known devices of the above-mentioned type generally give satisfaction, but they suffer from a certain number of problems, and in particular problems of tolerance by the patient.

It turns out to be particularly important to reduce as much as possible the sensation of discomfort such bands produce in the zone where the stomach is restricted, and to avoid or reduce the appearance of cell lesions in the restriction zone.

Unfortunately, for reasons of design and in particular of strength, known gastroplasty bands are always a source of discomfort or cell inflammation or lesions in the restriction zone. In order to ensure that such bands are strong enough, and in particular to ensure that the closure of the band is reliable, it turns out to be necessary to use elastomer materials having a high degree of hardness on the Shore A scale, and thus considerable rigidity, and although that does indeed contribute to strengthening the band, it also contributes to turning it into a source of trauma for the cell tissue and for the patient.

In particular, it turns out that although positioning the closure means on the dorsal portion of the band makes it possible to obtain a band whose adjustable annular portion surrounds the stomach over 360°, which reduces tissue trauma, it nevertheless contributes to exerting particularly large opposing traction forces on the outer closure means, which need specifically to be accommodated by reinforcing the general stiffness of the band. The design of known prior art bands is thus a result of a large number of technical compromises that are difficult to master, and that always lead to a certain amount of trauma for the patient.

It also turns out that the fabrication methods used for making such gastroplasty bands are difficult to implement since they generally involve fabricating dorsal reinforcement for the band together with the closure means, with the annular chamber proper of the band being bonded to the dorsal reinforcement. It will be understood that that type of method can lead to non-negligible risks of the parts that have been bonded together separating, at least in part, and that some number of defective items will be detected during fabrication. Known devices and methods therefore generally turn out to be difficult to implement and of relatively high industrial cost, if it is desired to obtain items that present good regularity and that are free from any defects.

### SUMMARY OF THE INVENTION

Consequently, the object given to the invention is to propose a novel gastroplasty band that makes it possible to remedy the various drawbacks set out above and that is particularly non-traumatic and well tolerated by the patient, while nevertheless being robust, easy to fabricate, and of low cost.

Another object of the invention seeks to propose a novel gastroplasty band that is particularly reliable in its mechanical behavior.

Another object of the invention is to propose a novel gastroplasty band that is particularly robust while also being particularly non-traumatic.

Another object of the invention seeks to propose a novel gastroplasty band in which the closure means are particularly strong.

Another object of the invention seeks to propose a novel gastroplasty band that is particularly simple to fabricate.

The object given to the invention seeks also to propose a novel method of fabricating a gastroplasty band by injecting an elastomer material into a mold, said novel method being particularly simple and fast, while nevertheless enabling a gastroplasty band to be obtained that is robust and non-traumatic.

Another object of the invention seeks to propose a novel fabrication method that is particularly inexpensive and that enables the number of fabrication steps to be small.

Another object of the invention seeks to propose a novel fabrication method that is particularly adapted to making a one-piece gastroplasty band.

Another object of the invention seeks to propose a novel fabrication method enabling a gastroplasty band to be obtained that is particularly reliable and safe.

The objects given to the invention are achieved by means of a gastroplasty band formed by a flexible strip designed to be closed around the stomach of a patient by closure means towards the two ends of the strip in order to reduce the diameter of the opening of the stoma, said strip including an annular compression chamber of adjustable volume connected by a catheter to a device for adjusting the diameter of said chamber by injecting or withdrawing fluid, said chamber being defined by walls comprising dorsal reinforcement extended by lateral walls, the band being characterized in that:
the dorsal reinforcement is made out of a first elastomer material having predetermined hardness d₁ on the Shore A scale; and
the lateral walls are made out of a second elastomer material of the same kind as the first material but of predetermined hardness d₂ on the Shore A scale that is such that d₂<d₁, thereby obtaining a one-piece annular chamber of hardness on the Shore A scale that varies across its thickness.

The objects given to the invention are also achieved by means of a method of fabricating a gastroplasty band by injecting an elastomer material in a mold provided with at least one cavity having at least one core, the method being characterized by the steps of;
a) injecting a first elastomer material of predetermined hardness d₁ on the Shore A scale in order to make at least the dorsal reinforcement of the band; and
b) overmolding at least on the dorsal reinforcement by injecting a second elastomer material of the same kind as the first material but of predetermined hardness d₂ on the Shore A scale such that d₂<d₁, in order to make the remaining portions of the band and obtain an overmolded one-piece band of varying hardness.

### BRIEF DESCRIPTION OF THE DRAWINGS

Other objects and advantages of the invention will appear better on reading the following description and from the accompanying drawings given purely for illustrative and informative purposes, and in which:

FIG. 1 is a side view of a gastroplasty band in accordance with the invention in its open position;

FIG. 2 is a side view identical to that of FIG. 1 showing a gastroplasty band in accordance with the invention in its closed position;

FIG. 3 is a cross-section view showing a gastroplasty band in accordance with the invention in its closed position;

FIG. 4 is a diagrammatic view showing a step in the fabrication method in accordance with the invention, in which the dorsal reinforcement of the band is made by means of a cavity having a dorsal reinforcing core placed therein;

FIG. 5 shows a step of the fabrication method of the invention in which a portion of the band fixing means is made in a mold cavity for the closure means in which a ring core is placed;

FIG. 6 shows a step of the fabrication method in accordance with the invention in which a second elastomer material is overmolded on the dorsal reinforcement and the band ring cone in a band mold for obtaining the final band;

FIG. 7 is a perspective view showing the dorsal reinforcing core supporting the dorsal reinforcement of the band as obtained at the end of the injection step shown in FIG. 4;

FIG. 8 is a perspective view showing the ring core supporting the reinforcing ring as obtained at the end of the injection step shown in FIG. 5; and

FIG. 9 is a cross-section view showing the particular shape of the section of the dorsal zone of the band.

### BEST MANNER OF PERFORMING THE INVENTION

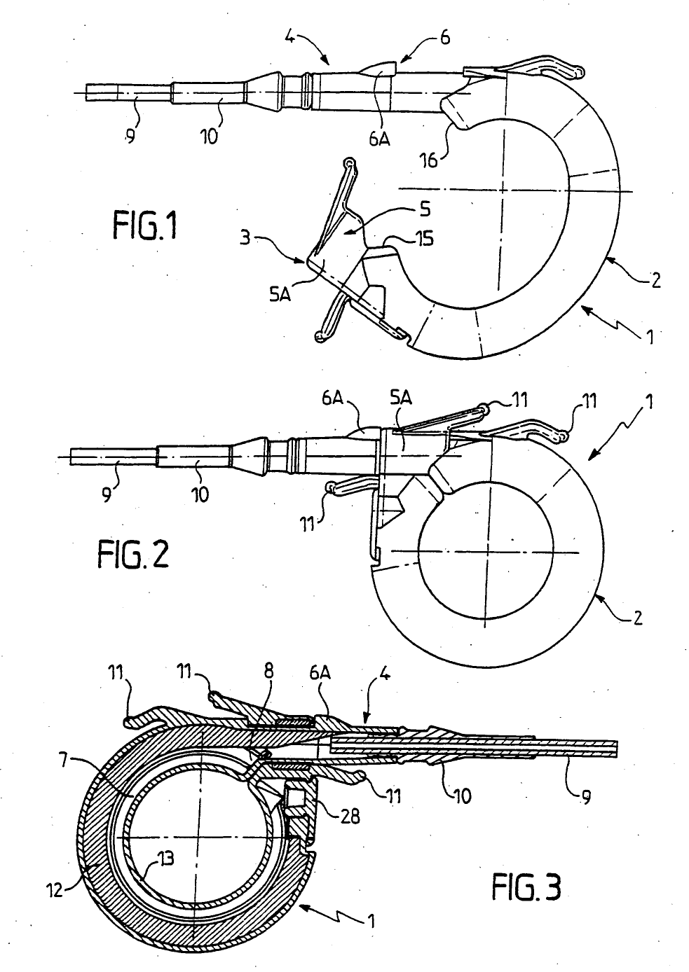

FIGS. 1 to 3 show a gastroplasty band 1 in accordance with the invention formed by a flexible strip 2 made of elastomer material, e.g. silicone, that is designed to be closed around the stomach of a patient by closure means 5, 6 located substantially towards its two ends 3, 4 for the purpose of reducing the diameter of the opening of the stoma.

The closed position of the band is shown in FIG. 2, in which position the closure means 5, 6 co-operate mutually to lock the band 1.

The band 1 in accordance with the invention also comprises, internally, a compression chamber 7 extending over the major fraction of the length of the flexible strip 2 in such a manner that in the closed position it forms an annular compression chamber 7 suitable for clamping the stomach around an angular range equal or substantially equal to 360°.

In known manner, the annular chamber 7 is of adjustable volume, i.e. its diametral expansion can be adjusted in expansion or in retraction so as to adjust correspondingly the diameter of the opening of the stoma. For this purpose, the annular compression chamber 7 is connected via the opening 8 and a catheter 9 associated with the opening 8 to a device (not shown in the figures) for adjusting the diameter of said chamber by injecting or withdrawing fluid. In known manner, the adjustment device is constituted by a miniature housing that can be implanted beneath the skin of the patient, the housing including a self-closing membrane that is designed to be pierced by a syringe enabling a certain quantity of fluid (generally physiological water) to be injected or withdrawn, thereby varying the volume of the annular compression chamber 7.

As shown in FIGS. 1 to 3, the catheter 9 can be connected to the flexible strip 2 via a connection member such as an endpiece 10. The gastroplasty band 1 in accordance with the invention may be provided with one or more grips 11 disposed at predetermined locations, e.g. towards the ends 3, 4 so as to make the band easier to handle, and in particular easier to close, and above all to make it easier to open or unlock.

As is also known, the gastroplasty band 1 in accordance with the invention includes a chamber 7 which is defined by walls comprising dorsal reinforcement 12 extended on either side towards the geometrical center of the closed band by lateral walls 13 which are advantageously of thickness that is smaller than the thickness of the dorsal reinforcement 12. This provides a gastroplasty band 1 in which the rigidity of the outer dorsal portion of the band 1 is greater than that of the inner portion of the band that comes into contact with stomach tissues.

Advantageously, the dorsal reinforcement is substantially of channel section, with the web 12A of the channel forming the typically dorsal portion of the band and being of thickness that is greater than the thickness of the flanges 12B of the channel (FIG. 9).

According to important characteristics of the invention, the dorsal reinforcement 12 is made out of a first elastomer material of predetermined hardness d1 on the Shore A scale, whereas the lateral walls 13 are themselves made of a second elastomer material of the same kind as the first material but of predetermined hardness d2 on the Shore A scale such that d2<d1, thereby obtaining a one-piece annular chamber 7 of Shore A hardness that varies across the thickness of its walls.

In the meaning of the invention, the term “elastomer material of the same kind” is used to mean a material of chemical composition that is very close, or similar, or identical, the materials differing from each other significantly only by their characteristic stiffness or rigidity.

By using materials of different hardnesses, better control is obtained over the deformation of the annular chamber 7, in particular for its inner portion that comes into contact with the stomach, so that the stomach is compressed in a manner that is particularly gentle and well tolerated. Otherwise, the band 1 that is obtained is particularly strong since the major portion of the mechanical traction forces is taken up by the dorsal reinforcement 12 which supports the closure means 5, 6 and which is of greater rigidity.

The combined effect of dorsal reinforcement and an annular chamber made of elastomer having different hardness also makes it possible to obtain an adjustment range on the inside diameter of the band that is large, with the advantage of simplifying band selection by reducing options to a single size.

By using two elastomer materials of the same kind, it is possible to obtain the annular chamber 7 by an operation of overmolding the two elastomer materials, the lateral walls 13 being overmolded on the dorsal reinforcement 12. This produces excellent continuous cohesion between the elastomer materials of the same kind.

As shown in FIG. 9, the flanges 12B facilitate bonding during overmolding with the material of smaller hardness that forms the lateral walls 13, while also facilitating positioning of the parts during overmolding. The specific channel-section shape of the dorsal reinforcement 12 with its web 12B forming an extra thickness of material of increased hardness, combined with thin lateral walls 13 of smaller hardness, leads to an annular compression chamber 7 being obtained in which compression is directed mostly if not exclusively centripetally (arrow F in FIG. 9), i.e. directed towards the geometrical center of the band. The chamber deforms above all via the lateral walls 13 of hardness and thickness that are small compared with the web 12B which deforms little or not at all.

By means of this technique, excellent bonding is obtained for the walls of the annular chamber 7 which ensures safety while also facilitating fabrication. It is also particularly easy to make a gastroplasty band 1 in accordance with the invention by using two simple overmolding steps corresponding to injecting two elastomer materials of the same kind so as to obtain a one-piece band of two overmolded materials with dorsal reinforcement made out of a first elastomer material having Shore A hardness d1 that is greater than the Shore A hardness of the second elastomer material constituting the remainder of the band, the lateral walls of the annular chamber 7, the closure means 5, 6, and advantageously the grips 11, the catheter 9, and the endpiece 10.

Advantageously, the value of d1 lies in the range 65 to 85 on the Shore A scale, with the value of d2 lying in the range 25 to 45 on the Shore A scale. In particularly advantageously manner, the value of d1 is about 80 on the Shore A scale and the value d2 is about 30 on the Shore A scale.

In a particularly advantageous version of the invention, the closure means 5, 6 are also made of elastomer materials and are disposed on the dorsal portion of the band, i.e. on the outside of the band when it is in its closed position, as shown in FIG. 2.

In known manner, the closure means 5, 6 comprise female means 5A secured to the end 3 of the flexible strip and formed by a pierced sleeve or a ring. The closure means also comprise male means 6A secured to the other end 4 of the flexible strip 2, the male means 6A being formed, for example, by a substantially radial projection constituting an abutment and by a zone that is suitable for expanding under the effect of an increase in the internal pressure inside the annular chamber 7.

In the closed position, the catheter 9 together with the endpiece 10 and the end 4 are inserted through the ring 5A so that the ring co-operates with the male means 6A to close the band 1.

In a particularly advantageous variant of the invention, at least a portion of the closure means 5, 6 is made out of the first elastomer material. In a particularly advantageous variant of the invention, the ring 5A is made from the first elastomer material presenting the greater hardness on the Shore A scale. By virtue of this feature, non-negligible reinforcement is obtained of the closure strength of the band, the portion 5A being made of a material presenting the higher rigidity, whereas the male means 6, 6A can be made out of the elastomer material presenting the lower hardness, given the need for the radial expansion it is to perform.

Advantageously, the thickness of the section of the dorsal reinforcement varies in substantially regular manner from one end to the other, and advantageously is at a maximum towards the portion 5A and at a minimum towards the means 6, 6A.

In another particularly advantageous version of the invention, the catheter 9 is secured to the endpiece 10 which is itself secured to the end 4 of the flexible strip 2, said endpiece being overmolded directly on the catheter 9.

Advantageously, the endpiece 10 is made of an elastomer material identical to the first elastomer material forming the dorsal reinforcement 12.

Since the closure means 5, 6 are secured to the ends 3, 4 of the flexible strip 2 and extend towards the outside of said strip away from the dorsal reinforcement 12, the annular chamber 7 is advantageously terminated by two substantially plane transverse sections 15, 16 that come to bear against each other when the band is in its closed position (FIG. 2), thereby forming an annular compression chamber 7 that provides compression over the entire periphery of the band, i.e. over about 360°.

In particularly advantageous manner, the gastroplasty band 1 in accordance with the invention is made with an annular chamber 7 that presents a cross-section that is substantially elliptical in shape. This feature enables the chamber to be given a relatively wide bearing surface, and in any case a bearing surface that is wider than that of conventional annular chambers of circular section, given the ease with which the chamber can deform elastically due to the presence of the particularly flexible second elastomer material. This ease of deformation, and the bearing surface that is relatively wide or in any event of increased area, makes it possible to reduce contact pressure between the stomach and the band because of the relative increase in contact area, thereby reducing aggression on stomach tissues. Advantageously, the elliptical cross-section is substantially constant over the entire developed length of the annular chamber 7.

In preferred manner, the gastroplasty band in accordance with the invention also presents substantially circular shape memory, so as to make it easier for the surgeon to put the band into position, since when the band is at rest in its open and loose position (FIG. 1), it is already in a quasi- or substantially-circular shape close to its final position as shown in FIG. 2.

Finally, as shown in particular in FIG. 3, the overmolding of the second elastomer material can lead to the presence of a small thickness of the lower-rigidity elastomer material (the second elastomer material) on and around the dorsal reinforcement 12.

The method of fabricating a gastroplasty band in accordance with the invention is a method involving injecting at least two (and in the present case only two) elastomer materials of identical kind into a mold provided with a cavity that itself includes at least one core.

According to the invention, the method of fabricating the band 1 is characterized by the steps of:
a) injecting a first elastomer material of predetermined hardness d₁ on the Shore A scale in order to make at least the dorsal reinforcement of the band; and
b) overmolding at least on the dorsal reinforcement by injecting a second elastomer material of the same kind as the first material but of predetermined hardness d₂ on the Shore A scale such that d₂<d₁, in order to make the remaining portions of the band and obtain an overmolded one-piece band of varying hardness.

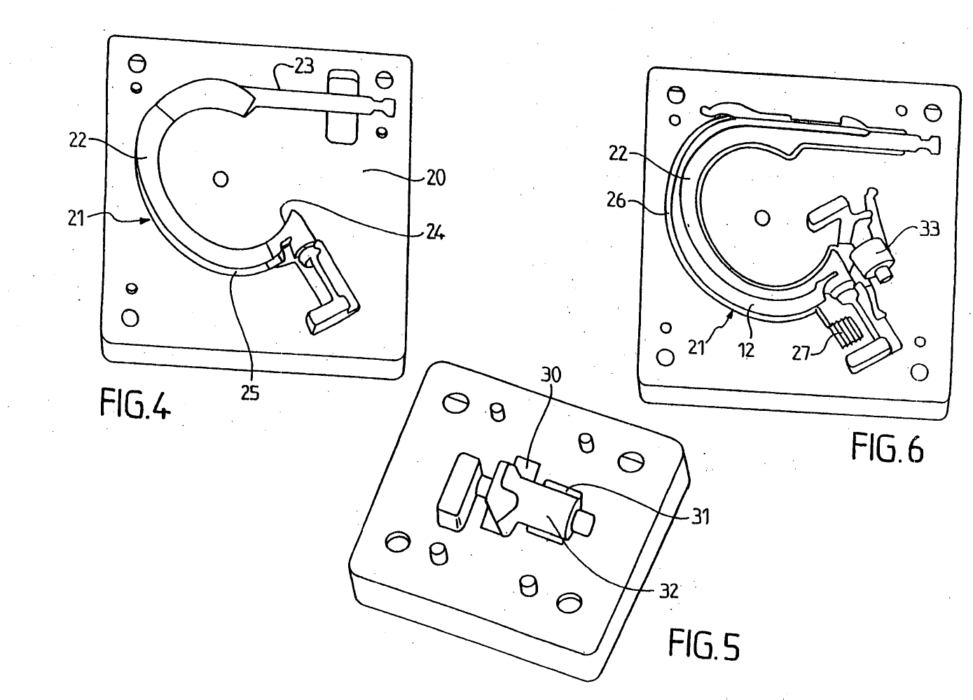

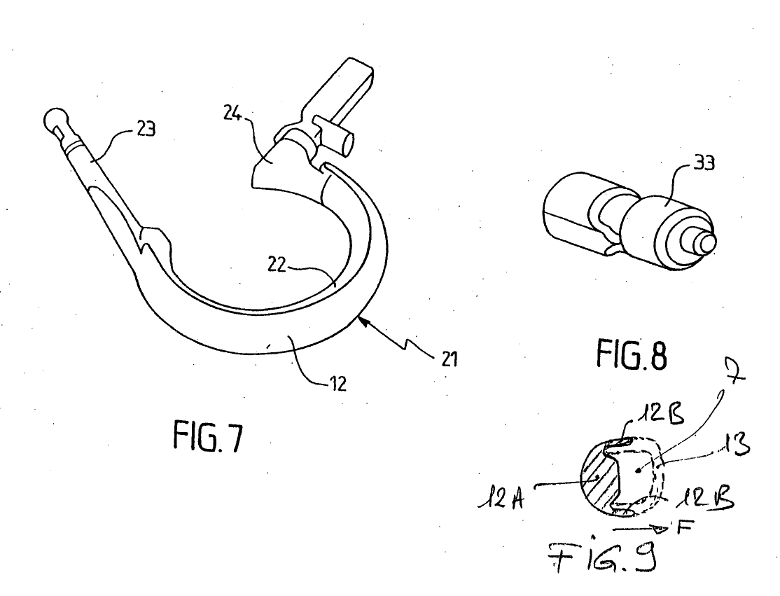

As shown in FIG. 4, during step a), the first elastomer material is injected into a dorsal cavity formed in a mold 20, which dorsal cavity receives a dorsal reinforcement core 21. As shown in FIG. 4, the dorsal reinforcement core 21 has a central portion 22 of substantially curved shape corresponding to the curved prestress desired for the dorsal reinforcement 12, and it has two end portions 23 and 24 corresponding respectively to the male and the female portions of the final band. The central portion 22 is substantially of channel section so as to form a groove 25 that is to receive the major fraction of the injected first elastomer material so as to form the main ridge of the dorsal reinforcement 12. The mold cavity is complementary in shape to the dorsal reinforcement core 21 so as to obtain, at the end of step a), a dorsal reinforcement core 21 supporting the dorsal reinforcement 12, as shown in FIG. 7. In this step, the dorsal reinforcement 12 extends over the major fraction of the outside surface of the core 21 and in particular in the groove 25, and it presents a channel section of thickness that varies in substantially regular manner from one end to the other. Advantageously, its thickness is greatest at or close to its end portion 24, and decreases regularly to its minimum value close to its end portion 23. The compression central portion nevertheless advantageously remains of constant thickness.

At the end of step a), the reinforcement core 21 supporting the dorsal reinforcement 12 (FIG. 7) is withdrawn and the reinforcement is placed in another mold (FIG. 6) having a cavity corresponding to the final shape of the complete band 1.

The fabrication method of the invention can then continue normally by overmolding the remainder of the band on the dorsal reinforcement 12 and on the dorsal reinforcement core 21 by injecting the second elastomer material so as to obtain the final one-piece band of varying rigidity (step b).

Advantageously, as shown in FIG. 6, the cavity for the final band 26 can include, preferably towards the dorsal portion of the band corresponding to the portion 24 of the core 21, a zone 27 of the cavity that is suitable for causing a plug 28 to be injected and formed (FIG. 3). The plug 28 that is obtained by the operation of overmolding the lower-rigidity elastomer material is integral with the body of the final band and serves to close in leaktight manner, e.g. using adhesive, the band in a loop, which band includes at this location an orifice due to the passage of tools enabling the final band and the dorsal reinforcement core 21 to be extracted at the end of step a).

In a particularly advantageous variant of the method of the invention, it is possible during step a) to make a portion of the band closure means out of the first elastomer material of greater hardness.

This specific step makes it possible to increase the strength of the final band and can be implemented in a distinct mold (FIG. 5), having a cavity formed therein corresponding to the closure means 30 comprising in particular a specific zone 31 such that when a ring core 32 is placed in the cavity 30, the zone 31 enables a ring 33 to be obtained, as shown in FIG. 8.

Thus, during step a), a portion of the closure means of the ring is made by injecting the first material of greater rigidity into a recess 30, 31 for the closure means, constituting a ring, and having a ring core 32 disposed therein.

Thereafter, prior to step b), the ring core 32 supporting the ring 33 (FIG. 8) is withdrawn and it is placed in the band recess 26 (FIG. 6) together with the core 21 for the dorsal reinforcement or intended to receive said dorsal reinforcement core. The dorsal reinforcement core 21 may already be in place in the recess for the final band, or on the contrary it may be put into place after the ring core 32 is put into place.

Thereafter, the second elastomer material can be injected so as to overmold the second material of lower rigidity onto the dorsal reinforcement 12 and the ring 33 made out of the more rigid elastomer material.

In this preferred variant, the ring 33 forms the essential portion of the female closure means 5A.

In another variant that is particularly advantageous, it is possible during step b) in which the second elastomer material is overmolded and injected, simultaneously to make the endpiece 10 for the catheter 9 which is thus made of the second elastomer material of lower rigidity.

This overmolding operation (not shown in the figures) is implemented in conventional manner in a specific mold having a suitable cavity. Thus, with the catheter 9 placed in the cavity for the endpiece, the second elastomer material is injected so as to obtain an endpiece 10 overmolded on the catheter 9.

Thereafter, the catheter 9 supporting the endpiece 10 is withdrawn and said endpiece is assembled, e.g. by adhesive, with the final band obtained at the end of step b), as shown in FIG. 6, for example.

The method as described above makes it possible to obtain very good bonding between the overmolded elastomer materials, and enables it to be achieved in a relatively short length of time, with the set of operations being easily automated and requiring a minimum of manual operations.

A one-piece band is also obtained that presents great regularity in all of the elements that make it up.

The invention also provides a method of therapeutic treatment of morbid obesity including the steps of installing, checking, and adjusting in particular the diameter of a gastric band in accordance with the invention.

### SUSCEPTIBILITY OF INDUSTRIAL APPLICATION

Industrial application of the invention lies in the design and fabrication of gastric bands for treating obesity.

1. A gastroplasty band (1) comprising:
a flexible strip (2) designed to be closed around the stomach of a patient by closure means (5, 6) towards the two ends (3, 4) of the flexible strip in order to reduce the diameter of the opening of the stoma, said flexible strip including an annular compression chamber (7) of adjustable volume connected by a catheter (9) to a device for adjusting the diameter of said annular compression chamber by injecting or withdrawing fluid, said annular compression chamber being defined by walls comprising a dorsal reinforcement (12) extended by lateral walls (13);
wherein the dorsal reinforcement (12) is made out of a first elastomer material having predetermined hardness d₁ on the Shore A scale; and
wherein the lateral walls (13) are made out of a second elastomer material of the same kind as the first elastomer material but of predetermined hardness d₂ on the Shore A scale such that d₂<d₁, thereby obtaining the annular compression chamber (7) of hardness on the Shore A scale that varies across its thickness.

2. The gastroplasty band according to claim 1, wherein the annular compression chamber (7) is obtained by an operation of overmolding the two elastomer materials, the lateral walls (13) being overmolded on the dorsal reinforcement (12).

3. The gastroplasty band according to claim 1, wherein the value of d₁ lies in the range 65 to 85 on the Shore A scale, with the value of d₂ lying in the range 25 to 45 on the Shore A scale.

4. The gastroplasty band according to claim 3, wherein the value of d₁ is about 80 on the Shore A scale, and the value of d₂ is about 30 on the Shore A scale.

5. The gastroplasty band according to claim 1, wherein a portion of the closure means (5, 6) is made out of the first elastomer material.

6. The gastroplasty band according to claim 5, wherein the closure means (5, 6) comprise female means (5A) secured to one end (3) of the flexible strip (12) to co-operate with male means (6) secured to the other end of the flexible strip, the female means being formed by a ring (5A) made out of the first elastomer material.

7. The gastroplasty band according to claim 1, wherein the catheter (9) includes an endpiece (10) secured to one end of the flexible strip, said endpiece being overmolded on the catheter (9).

8. The gastroplasty band according to claim 7, wherein the endpiece (10) is made out of the first elastomer material.

9. The gastroplasty band according to claim 1, wherein the closure means (5, 6) are secured to the ends (3, 4) of the flexible strip (2) and extend outwards from the strip from the dorsal reinforcement (12), the annular chamber (7) being terminated by two transverse sections (15, 16) that are substantially plane so as to bear against each other in the closed position of the band, thereby forming the annular compression chamber (7) providing compression over the entire periphery of the band.

10. The gastroplasty band according to claim 1, wherein the annular compression chamber (7) presents a cross-section that is substantially elliptical in shape.

11. The gastroplasty band according to claim 1, wherein the gastroplasty band presents shape memory that is substantially circular.

12. The gastroplasty band according to claim 1, wherein the dorsal reinforcement (12) presents a shape that is substantially of channel section.

13. The gastroplasty band according to claim 12, wherein the dorsal reinforcement (12) comprises a web (12A) and flanges (112B), the web (12A) having a thickness greater than the thickness of the flanges (12B).

14. A method of fabricating a gastroplasty band by injecting an elastomer material in a mold provided with at least one cavity having at least one core, the method comprising:
a) injecting a first elastomer material of predetermined hardness d₁ on the Shore A scale in order to make at least a dorsal reinforcement of the gastroplasty band; and
b) overmolding at least on the dorsal reinforcement by injecting a second elastomer material of the same kind as the first elastomer material but of predetermined hardness d₂ on the Shore A scale such that d₂<d₁, in order to make the remaining portions of the gastroplasty band and obtain an overmolded one-piece gastroplasty band of varying hardness.

15. The method according to claim 14, further comprising:
during step a), making a portion of closure means of the band out of the first elastomer material.

16. The method according to claim 14, further comprising:
making a catheter endpiece out of the second elastomer material.

17. The method according to claim 14, further comprising:
during step a), injecting the first elastomer material into a dorsal cavity having a dorsal reinforcement core placed therein;
withdrawing the dorsal reinforcement core supporting the dorsal reinforcement and placing it in a band recess; and
performing step b) to obtain the final band.

18. The method according to claim 17, further comprising:
during step a), making a portion of fixing means of the band by injecting the first material in a cavity for ring-shaped fixing means having a ring core placed therein;
prior to step b), withdrawing the ring core supporting the ring and placing it in said band cavity together with the dorsal reinforcing core.

19. The method according to claim 16, further comprising:
during step b), making the catheter endpiece by injecting the second elastomer material into a catheter cavity including a catheter;
removing the catheter supporting the catheter endpiece; and assembling said catheter endpiece with the gastroplasty band obtained at the end of step b).

20. The method according to claim 19, wherein assembling said endpiece with the gastroplasty band comprises applying an adhesive.
